(19) United States
(12) Patent Application Publication
(10) Pub. No.: US 2024/0132777 A1
(43) Pub. Date: Apr. 25, 2024

(54) DYES FOR NUCLEIC ACID DETECTION

(71) Applicant: Detect, Inc., Guilford, CT (US)

(72) Inventor: Roger R. Nani, Madison, CT (US)

(73) Assignee: Detect, Inc., Guilford, CT (US)

(21) Appl. No.: 18/482,816

(22) Filed: Oct. 6, 2023

Related U.S. Application Data
(60) Provisional application No. 63/414,618, filed on Oct. 10, 2022.

(51) Int. Cl. C09K 11/06; C09B 23/04

(52) U.S. Cl. CPC C09K 11/06; C09B 23/04; C09K 2211/1007; C09K 2211/1018

(57) ABSTRACT

Provided herein are dye compounds having improved properties, such as aqueous solubility, stability, and fluorescence, as well as methods of detecting the presence or absence of an analyte in a test sample using said dye compounds.

### CROSS-REFERENCE TO RELATED APPLICATIONS

The present application claims the benefit of priority of U.S. Provisional Application No. 63/414,618 filed Oct. 10, 2022, entitled “DYES FOR NUCLEIC ACID DETECTION” (Attorney Docket No. H0966.70085US00), the entire contents of which is incorporated by reference herein.

### BACKGROUND OF THE INVENTION

Dyes are commonly used in the biotechnological and biomedical research fields for the detection of molecules of interest, such as nucleic acids. The detection and quantification of nucleic acids, particularly DNA, using fluorescent dyes in biological test samples is an important tool in the diagnosis of various genetic or pathological conditions. As more sophisticated diagnostic and testing platforms are developed, such as desktop microfluidic devices, there is an increased need for dye compounds having properties amenable to the new mechanical and chemical requirements of the platforms.

### SUMMARY OF THE INVENTION

Accordingly, provided herein are dye compounds having improved properties, such as aqueous solubility, stability, and fluorescence. In one aspect, provided herein is a compound of formula (I):

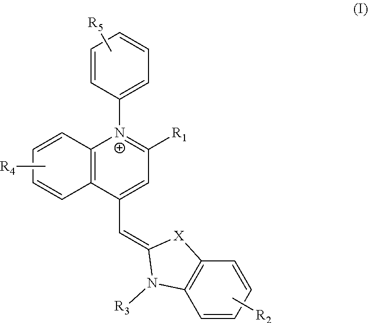

In certain embodiments, the compound of formula (I) is conformationally and/or configurationally stable upon intercalation into double-stranded DNA, such as to emit a useful fluorescence signal upon irradiation.

In certain embodiments, the emission spectrum of complexes comprising the compound of formula (I) are characterized as having a favorable signal-to-noise ratio. In certain embodiments, the compound of formula (I) provides minimal inhibition of isothermal nucleic acid amplification processes. In certain embodiments, the compound of formula (I) is characterized as providing sufficient endpoint intensity for the detection of nucleic acid (e.g., double stranded DNA) analytes.

In another aspect, provided herein is a method of detecting the presence or absence of an analyte in a test sample, comprising contacting the test sample with a compound of formula (I) in the presence of an energy (E₁) and detecting emission of an energy (E₂) from the sample.

### BRIEF DESCRIPTION OF THE DRAWINGS

FIGS. 1A-1C depict fluorescence data for compounds 7 and 8 both in the presence and in the absence of DNA. FIG. 1A depicts data for compound 7. FIG. 1B depicts data for compound 8. FIG. 1C depicts a comparison of the fluorescence of compounds 7 and 8 at 505 nm.

### DETAILED DESCRIPTION OF CERTAIN EMBODIMENTS OF THE INVENTION

In one aspect, provided herein is a compound comprising formula (I):

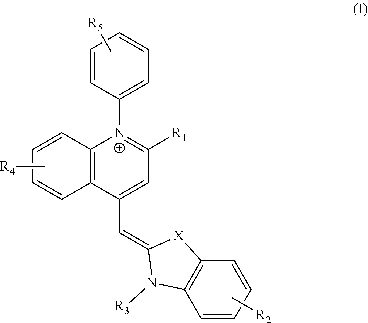

wherein:

X is O, or S;

R₁ is C₁₋₆ alkyl optionally substituted with a polyethylene glycol moiety;

R₂ is C₁₋₆ alkyl optionally substituted with a polyethylene glycol moiety, a polyethylene glycol moiety, or is absent;

R₃ is C₁₋₆ alkyl optionally substituted with a polyethylene glycol moiety;

R₄ is C₁₋₆ alkyl optionally substituted with a polyethylene glycol moiety, a polyethylene glycol moiety, or is absent;

R₅ C₁₋₆ alkyl optionally substituted with a polyethylene glycol moiety, a polyethylene glycol moiety, or is absent;

provided that the compound comprises at least one polyethylene glycol moiety.

In certain embodiments, the compound of formula (I) further comprises an anion to achieve electronic neutrality. Suitable anions include, but are not limited to, anions of alkali metals (e.g., Li, Na, and K), alkali earth metals (e.g., Mg and Ca), borates, boronates, and carboxylates. In a particular embodiment, the compound of formula (I) further comprises an acetate anion.

In certain embodiments, the polyethylene moiety is of formula (i):

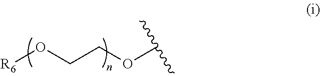

wherein:

n is 1-10, inclusive; and

R₆ is optionally substituted aliphatic.

In certain embodiments, R₆ is C₁₋₆ alkyl. In a particular embodiment, R₆ is methyl.

In certain embodiments, X is O. In certain embodiments, X is S.

In certain embodiments, R₁ is C₁₋₆ alkyl substituted with a moiety of formula (i). In certain embodiments, R₂, R₃, R₄, and R₅ do not comprise a polyethylene glycol moiety.

In certain embodiments, R₂ is C₁₋₆ alkyl substituted with a moiety of formula (i). In certain related embodiments, R₁, R₃, R₄, and R₅ do not comprise a polyethylene glycol moiety.

In certain embodiments, R₃ is C₁₋₆ alkyl substituted with a moiety of formula (i). In certain related embodiments, R₁, R₂, R₄, and R₅ do not comprise a polyethylene glycol moiety.

In certain embodiments, R₄ is C₁₋₆ alkyl substituted with a moiety of formula (i). In certain related embodiments, R₁, R₂, R₃, and R₅ do not comprise a polyethylene glycol moiety.

In certain embodiments, R₅ is a moiety of formula (i). In certain related embodiments, R₁, R₂, R₃, and R₄ do not comprise a polyethylene glycol moiety.

In certain embodiments, the compound of formula (I) has the structure of formula (II):

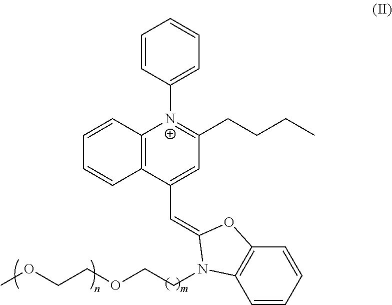

wherein n is 1-10, inclusive; and m is 1-5, inclusive. In certain embodiments, the compound of formula (II) further comprises an acetate anion.

In certain embodiments, the compound of formula (I) has the structure of formula (III):

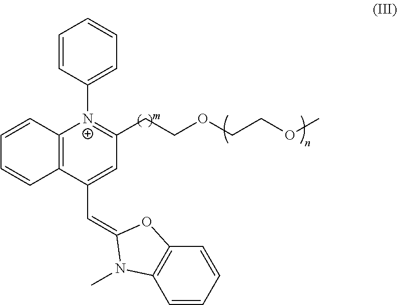

wherein n is 1-10, inclusive; and m is 1-5, inclusive. In certain embodiments, the compound of formula (III) further comprises an acetate anion.

In certain embodiments, the compound of formula (I) has the structure of formula (IV):

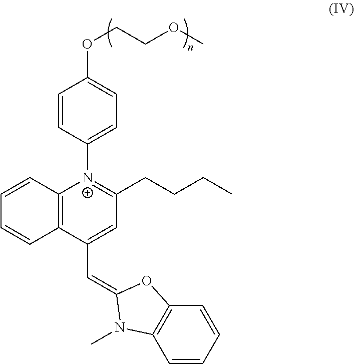

wherein n is 1-10, inclusive. In certain embodiments, the compound of formula (IV) further comprises an acetate anion.

In certain embodiments, the compound of formula (I) has the structure of formula (V):

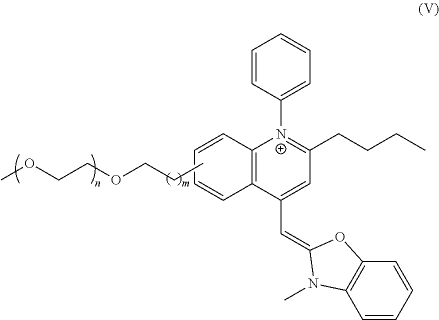

wherein n is 1-10, inclusive; and m is 1-5, inclusive. In certain embodiments, the compound of formula (V) further comprises an acetate anion.

In certain embodiments, the compound of formula (I) has the structure of formula (VI):

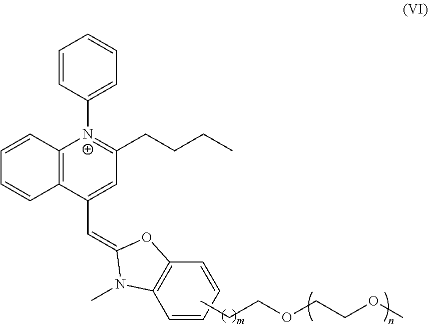

wherein n is 1-10, inclusive; and m is 1-5, inclusive. In certain embodiments, the compound of formula (VI) further comprises an acetate anion.

In certain embodiments, the compound of formula (I) has a structure selected from:

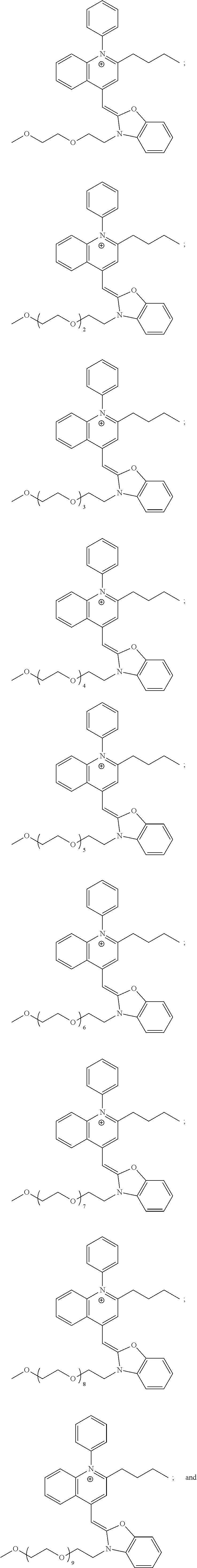

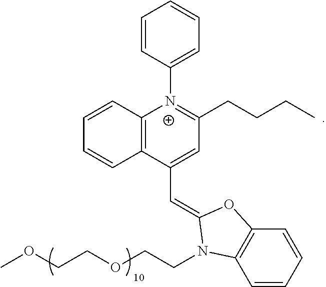

In certain embodiments, the preceding compounds further comprises an acetate anion.

In another aspect, provided herein is a composition comprising a compound of formula (I), in any of the several embodiments disclosed herein.

In certain embodiments, the composition is a solution or a suspension. In certain embodiments, the concentration of the compound is in the range of 0.001-100 μM. In certain embodiments, the concentration is in the range of 0.001-0.01 μM. In certain embodiments, the concentration is in the range of 0.01-0.1 μM. In certain embodiments, the concentration is in the range of 0.1-1 μM. In certain embodiments, the concentration is in the range of 1-10 μM. In certain embodiments, the concentration is in the range of 10-100 μM.

In certain embodiments, the composition further comprises a test sample. In certain embodiments, the test sample is of an animal bodily tissue or fluid.

In certain embodiments, the animal is a human. In certain embodiments, the test sample comprises an analyte. In certain embodiments, the analyte is a nucleic acid. In certain embodiments, the nucleic acid is DNA. In certain embodiments, the DNA is double-stranded DNA. In certain embodiments, the nucleic acid is of a virus or bacterium. In certain embodiments, the virus is SARS-CoV-2.

In another aspect, provided herein is a method of detecting the presence or absence of an analyte in a test sample, comprising contacting the test sample with a compound of formula (I), in any of the several embodiments disclosed herein, in the presence of an energy (E₁) and detecting emission of an energy (E₂) from the sample.

In certain embodiments, the test sample is of an animal bodily tissue or fluid. In certain embodiments, the animal is a human.

In certain embodiments, the analyte is a nucleic acid. In certain embodiments, the nucleic acid is DNA. In certain embodiments, the DNA is double-stranded DNA. In certain embodiments, the nucleic acid is of a virus or bacterium. In certain embodiments, the virus is SARS-CoV-2.

In certain embodiments, E₁ is electromagnetic radiation having a wavelength of about 400 nm to about 800 nm.

In certain embodiments, E₂ is emitted by fluorescence of the compound. In certain embodiments, E₂ is lower when the analyte is present. In certain embodiments, E₂ is higher when the analyte is present.

In certain embodiments, the test sample is a solution or suspension. In certain embodiments, the compound is present in an amount of 1-10 nmol per milliliter of test sample.

In another aspect, provided herein is a system for the detection of the presence or absence of double-stranded DNA comprising the use of a compound of formula (I) (e.g., formulae (II), (III), (IV), (V), or (VI)) and/or a method as described herein.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Unless provided otherwise, the term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

Unless provided otherwise, the term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F , —CH₂CHF₂, —CH₂CF₃, or benzyl (Bn)).

### EXAMPLES

In order that the invention described herein may be more fully understood, the following examples are set forth. It should be understood that these examples are for illustrative purposes only and are not to be construed as limiting this invention in any manner.

### Example 1. Synthesis of Compound 7

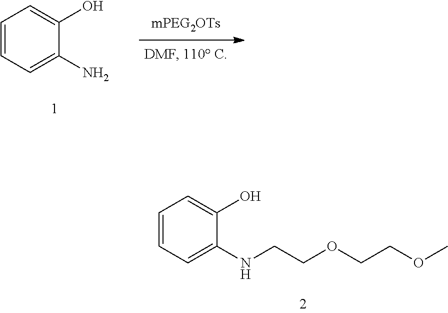

To a flask containing 2-aminophenol (1, 200 mg, 1.8 mmol) was added DMF (3 mL) under nitrogen. 2-(2-methoxyethoxy)ethyl 4-methylbenzenesulfonate (502 mg, 1.8 mmol) was added and the reaction was heated to 110° C. The reaction was monitored by HPLC until the mPEG₂OTs reactant was fully consumed. The reaction was cooled to room temperature, diluted with water (10 mL), and directly purified by reversed phase chromatography (0 →100% MeCN/H₂O 0.1% TFA, Hamilton PRP C₁₈21.1×250 mm column). Evaporation of the product containing fractions afforded 2 (116 mg, 30% yield) as a yellow oil. HRMS (ESI) calculated for C₁₁H₁₈NO₃ (M+H)⁺212.1281, observed 212.1301.

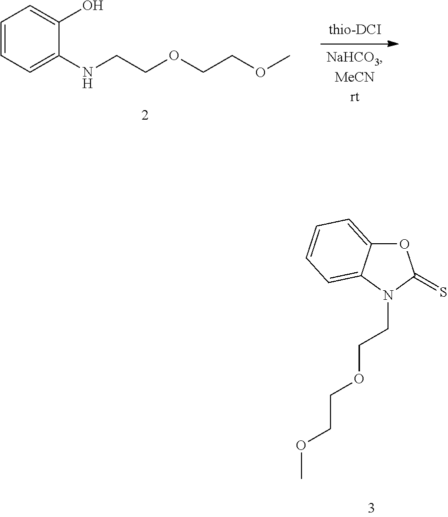 [SMILES: COCCOCCNc1ccccc1O ; COCCOCCn1c(=S)oc2ccccc21]

A flask was charged with 2-((2-(2-methoxyethoxy)ethyl)amino)phenol (2, 116 mg, 0.55 mmol) and sodium bicarbonate (46 mg, 0.55 mmol) and flushed with nitrogen. Acetonitrile (2 mL) was added followed by portion-wise addition of 1,1′-thiocarbonyldiimidazole (147 mg, 0.83 mmol) at room temperature. The reaction was monitored by HPLC during the portion-wise addition of thio-CDI until complete consumption of 2 occurred. The reaction was diluted with water (5 mL) and directly purified by reversed phase chromatography (0→100% MeOH/H₂O 0.1% TFA, Hamilton PRP C₁₈21.1×250 mm column). Evaporation of the product containing fractions afforded 3 (111 mg, 80% yield) as a colorless oil. HRMS (ESI) calculated for C₁₂H₁₆NO₃S (M+H)⁺254.0845, observed 254.0901.

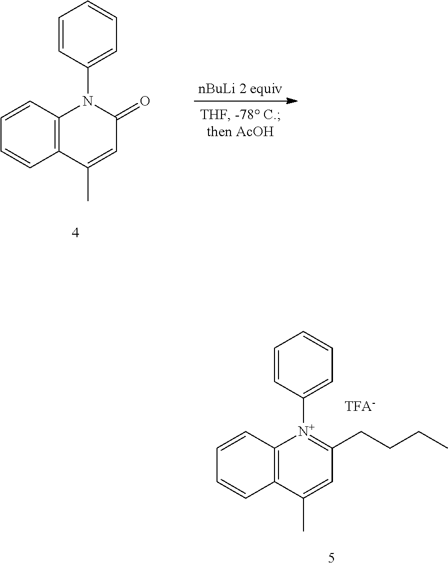 [SMILES: Cc1cc(=O)n(-c2ccccc2)c2ccccc12 ; CCCCc1cc(C)c2ccccc2[n+]1-c1ccccc1]

To a flask containing quinolone 4 (280 mg, 1.2 mmol) was added THF (5 mL) under nitrogen. The reaction was cooled to −78° C. and n-butyllithium (1.0 mL, 2.5 M hexanes) was added dropwise. The reaction was stirred at −78° C. for 1 hour and then warmed to 0° C. Acetic acid (0.2 mL) was added in one portion, and the reaction was warmed to room temperature. Water (5 mL) was added, and the reaction was directly purified by reversed phase chromatography (0 →100% MeCN/H2O 0.1% TFA, Hamilton PRP C18 21.1×250 mm column). Evaporation of the product containing fractions afforded 5 (420 mg, 90% yield) as a white solid. HRMS (ESI) calculated for C₂₀H₂N (M)+276.1747, observed 276.1755.

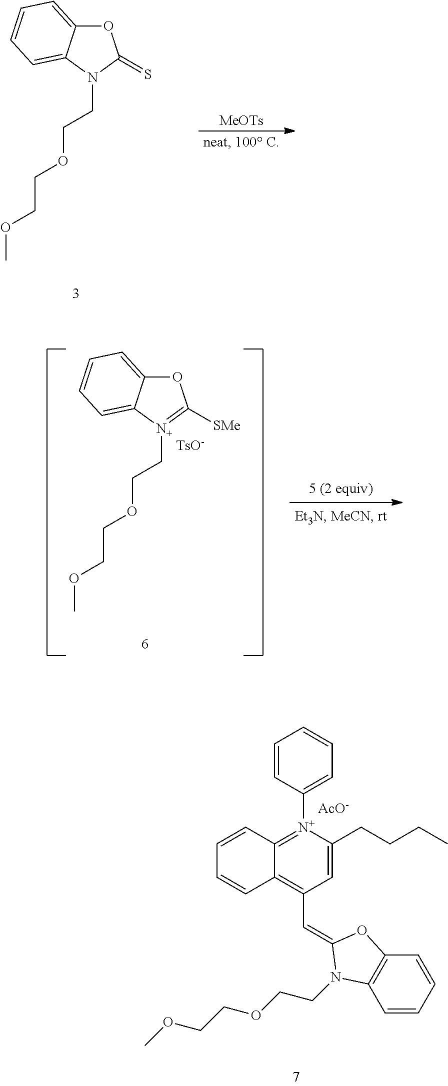

To a flask containing benzoxazole 3 (111 mg, 0.44 mmol) was added methyl p-toluenesulfonate (136 mL) under nitrogen. The reaction was heated to 100° C. with vigorous stirring. The reaction was monitored by HPLC until 90% conversion to intermediate 6 was achieved. The reaction was cooled to room temperature, and two equivalents of quinolinium 5 (342 mg, 0.88 mmol) in MeCN (1 mL) was added. Triethylamine (122 mL, 0.88 mmol) was added and the reaction was stirred at room temperature for 5 minutes. The mixture was diluted with water (0.5 mL) and directly purified by reversed phase chromatography (0 →100% MeCN/H₂O 0.1% TEAA, C₄ 21.1 ×50 mm column). Evaporation of the product containing fractions afforded 7 (122 mg, 50% yield) as a brown solid. HRMS (ESI) calculated for C₃₂H₃₅N₂O₃ (M)⁺495.2642, observed 495.2750.

### Example 2. Synthesis of Compound 8

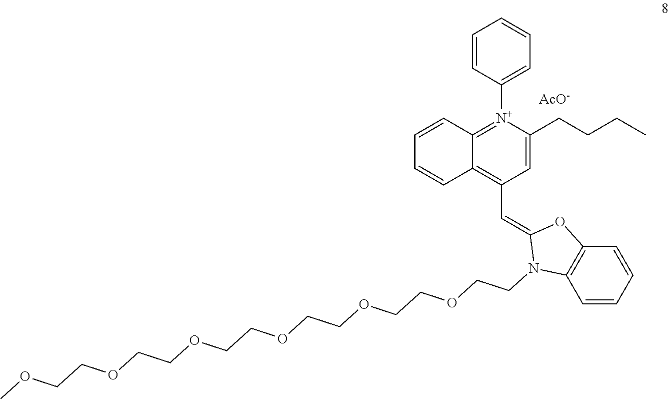 [SMILES: "AcO-" ; CCCCc1cc(/C=C2\Oc3ccccc3N2CCOCCOCCOCCOCCOCCOC)c2ccccc2[n+]1-c1ccccc1]

Compound 9 was prepared using a similar procedure to compound 7. HRMS (ESI) calculated for C₄₀H₅₁N₂O₇ (M)³⁰ 671.3691, observed 671.3799.

### Example 3. Retrosyntheses for Formulae (III), (IV), (V), (VI)

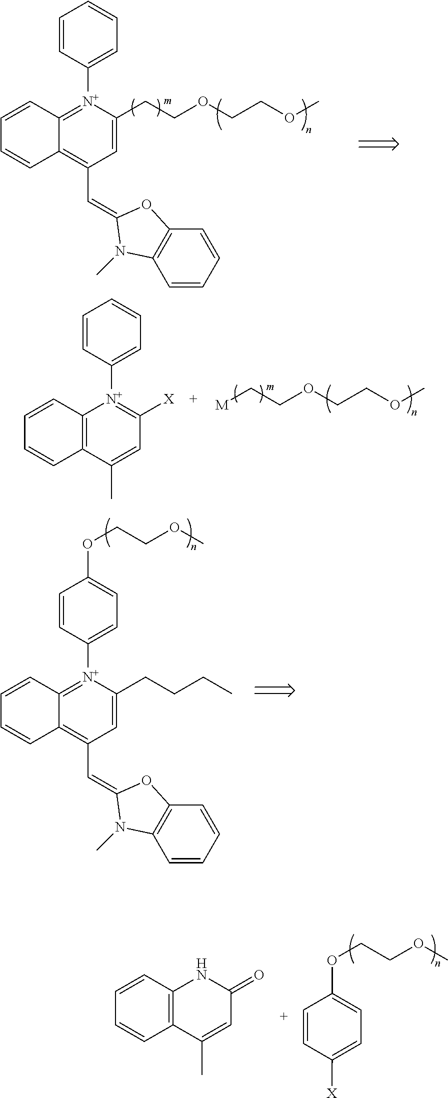

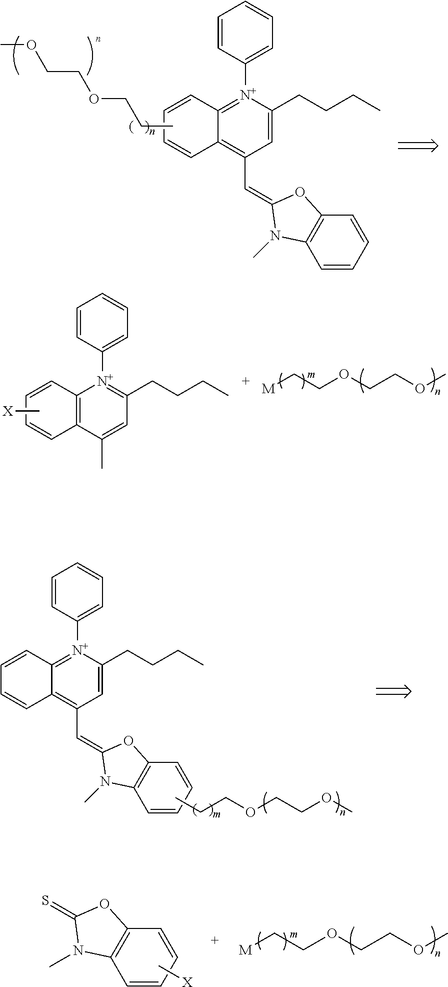

### Example 4. Procedure for Fluorescence Spectroscopy Study of Compounds 7 and 8

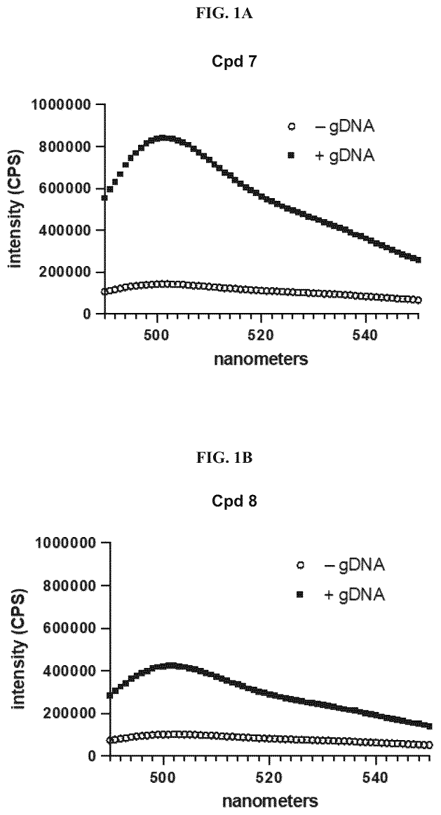

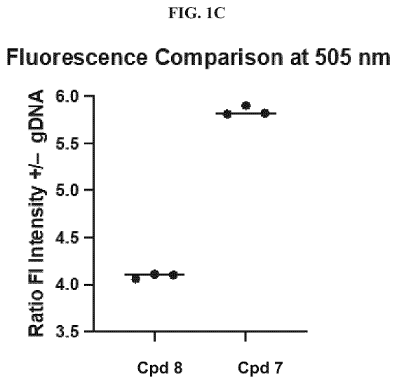

Solutions of compound 7 and compound 8 (500 nanomolar) were prepared in buffer A (20 mM Tris-HCl, 10 mM ammonium sulfate, 50 mM KCl, 8 mM MgSO₄, 0.2 mM ethylene glycol-bis(β-aminoelhyl ether)-N,N,N′,N′-tetraacetic acid, 0.1% Tween-20, pH 8). The separate solutions were placed in a quartz cuvettes and analyzed using a Fluorolog-QM-11 with Peltier controlled heating (Horiba. Scientific). The samples were excited at 450 nm (5 nm slit width) and the background corrected emission spectflun was collected from 490 to 550 nra slit width, 1 nm steps) at 63.5° C. 20 μg of E. coli genomic DNA (ThermoFisher) was added and the emission spectrum was recorded. The emission spectra for 7 and 8 +/− E. coli genomic DNA are plotted in FIGS. 1A and 1B. The difference in the ratio of the fluorescence intensity values at 505 nm +/− E. coli genomic DNA are plotted in FIGS. 1C.

### Claim Concepts

The following non-limiting concepts are disclosed herein.
1. A compound comprising formula (I):

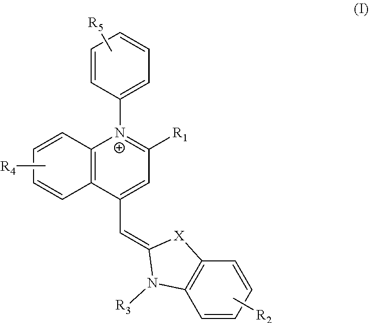

wherein:

X is O, or S;

R₁ is C₁₋₆ alkyl optionally substituted with a polyethylene glycol moiety;

R₂ is C₁₋₆ alkyl optionally substituted with a polyethylene glycol moiety, a polyethylene glycol moiety, or is absent;

R₃ is C₁₋₆ alkyl optionally substituted with a polyethylene glycol moiety;

R₄ is C₁₋₆ alkyl optionally substituted with a polyethylene glycol moiety, a polyethylene glycol moiety, or is absent;

R₅ C₁₋₆ alkyl optionally substituted with a polyethylene glycol moiety, a polyethylene glycol moiety, or is absent;

provided that the compound comprises at least one polyethylene glycol moiety.
2. The compound of claim 1, wherein the polyethylene moiety is of formula (i):

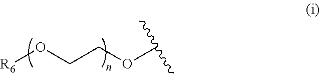

wherein:

n is 1-10, inclusive; and

R₆ is optionally substituted aliphatic.
3. The compound of claim 2, wherein R₆ is C₁₋₆ alkyl.
4. The compound of claim 3, wherein R₆ is methyl.
5. The compound of any one of claims 1-4, wherein X is O.
6. The compound of any one of claims 1-4, wherein X is S.
7. The compound of any one of claims 2-6, wherein R₁ is C₁₋₆ alkyl substituted with a moiety of formula (i).
8. The compound of claim 7, wherein R₂, R₃, R₄, and R₅ do not comprise a polyethylene glycol moiety.
9. The compound of any one of claims 2-6, wherein R₂ is C₁₋₆ alkyl substituted with a moiety of formula (i).
10. The compound of claim 9, wherein R₁, R₃, R₄, and R₅ do not comprise a polyethylene glycol moiety.
11. The compound of any one of claims 2-5, wherein R₃ is C₁₋₆ alkyl substituted with a moiety of formula (i).
12. The compound of claim 11, wherein R₁, R₂, R₄, and R₅ do not comprise a polyethylene glycol moiety.
13. The compound of any one of claims 2-6, wherein R₄ is C₁₋₆ alkyl substituted with a moiety of formula (i).
14. The compound of claim 13, wherein R₁, R₂, R₃, and R₅ do not comprise a polyethylene glycol moiety.
15. The compound of any one of claims 2-6, wherein R₅ is a moiety of formula (i).
16. The compound of claim 15, wherein R₁, R₂, R₃, and R₄ do not comprise a polyethylene glycol moiety.
17. The compound of claim 1 having the structure of formula (II):

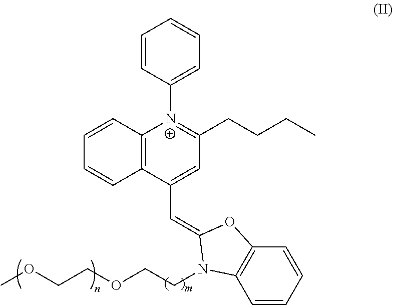

wherein n is 1-10, inclusive; and
m is 1-5, inclusive.
   18. The compound of claim 1 having the structure of formula (III):

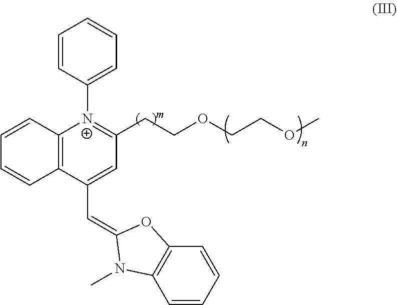

wherein n is 1-10, inclusive; and
m is 1-5, inclusive.
   19. The compound of claim 1 having the structure of formula (IV):

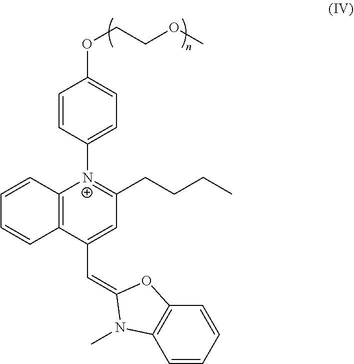

wherein n is 1-10, inclusive.
20. The compound of claim 1 having the structure of formula (V):

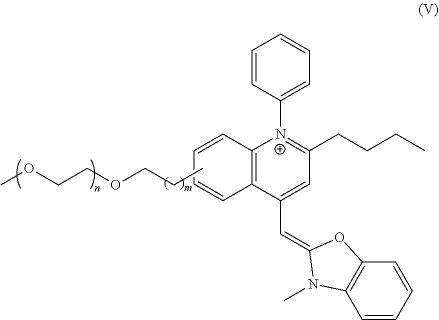

wherein n is 1-10, inclusive; and
m is 1-5, inclusive.
   21. The compound of claim 1 having the structure of formula (VI):

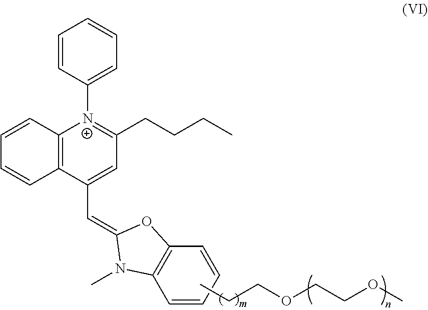

wherein n is 1-10, inclusive; and
m is 1-5, inclusive.
   22. The compound of claim 1, selected from:

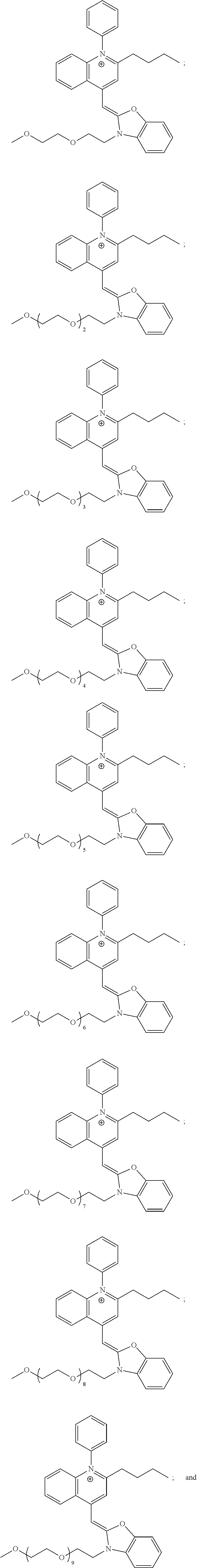

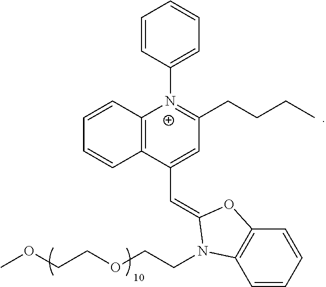

23. A composition comprising a compound of any one of claims 1-22.
24. The composition of claim 24, wherein the composition is a solution or a suspension.
25. The composition of claim 24, wherein the concentration of the compound is in the range of 0.001-100 μM.
26. The composition of any one of claims 23-25, wherein the composition further comprises a test sample.
27. The composition of claim 26, wherein the test sample is of an animal bodily tissue or fluid.
28. The composition of claim 27, wherein the animal is a human.
29. The composition of any one of claims 23-28, wherein the test sample comprises an analyte.
30. The composition of claim 29, wherein the analyte is a nucleic acid.
31. The composition of claim 30, wherein the nucleic acid is DNA.
32. The composition of claim 31, wherein the DNA is double-stranded DNA.
33. The composition of any one of claims 30-32, wherein the nucleic acid is of a virus or bacterium.
34. The composition of claim 33, wherein the virus is SARS-CoV-2.
35. A method of detecting the presence or absence of an analyte in a test sample, comprising contacting the test sample with a compound of any one of claims 1-22 in the presence of an energy (E₁) and detecting emission of an energy (E₂) from the sample.
36. The method of claim 35, wherein the test sample is of an animal bodily tissue or fluid.
37. The method of claim 36, wherein the animal is a human.
38. The method of any one of claims 35-37, wherein the analyte is a nucleic acid.
39. The method of claim 38, wherein the nucleic acid is DNA.
40. The method of claim 39, wherein the DNA is double-stranded DNA.
41. The method of any one of claims 38-40, wherein the nucleic acid is of a virus or bacterium.
42. The method of claim 41, wherein the virus is SARS-CoV-2.
43. The method of any one of claims 35-42, wherein E₁ is electromagnetic radiation having a wavelength of about 400 nm to about 800 nm.
44. The method of any one of claims 35-43, wherein E₂ is emitted by fluorescence of the compound.
45. The method of claim 44, wherein E₂ of the test sample is lower when the analyte is present.
46. The method of claim 44, wherein E₂ of the test sample is higher when the analyte is present.
47. The method of any one of claims 35-46, wherein the test sample is a solution or suspension.
48. The method of claim 47, wherein the compound is present in an amount of 1-10 nmol per milliliter of test sample.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub—range within the stated ranges in different embodiments of the invention, to the tenth of the unit of the lower limit of the range, unless the context clearly dictates otherwise.

This application refers to various issued patents, published patent applications, journal articles, books, manuals, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound comprising formula (I): 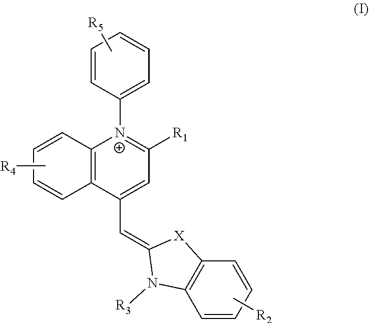
wherein:
X is O, or S;
R₁ is C₁₋₆ alkyl optionally substituted with a polyethylene glycol moiety;
R₂ is C₁₋₆ alkyl optionally substituted with a polyethylene glycol moiety, a polyethylene glycol moiety, or is absent;
R₃ is C₁₋₆ alkyl optionally substituted with a polyethylene glycol moiety;
R₄ is C₁₋₆ alkyl optionally substituted with a polyethylene glycol moiety, a polyethylene glycol moiety, or is absent;
R₅ C₁₋₆ alkyl optionally substituted with a polyethylene glycol moiety, a polyethylene glycol moiety, or is absent;
provided that the compound comprises at least one polyethylene glycol moiety.

2. The compound of claim 1, wherein the polyethylene moiety is of formula (i): 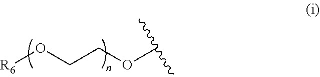
wherein:
n is 1-10, inclusive; and
R₆ is optionally substituted aliphatic.

3. The compound of claim 2, wherein R₆ is C₁₋₆ alkyl.

4. The compound of claim 3, wherein R₆ is methyl.

5. The compound of claim 1, wherein X is O.

6. The compound of claim 1, wherein X is S.

7. The compound of claim 2, wherein R₁ is C₁₋₆ alkyl substituted with a moiety of formula (i).

8. The compound of claim 7, wherein R₂, R₃, R₄, and R₅ do not comprise a polyethylene glycol moiety.

9. The compound of claim 2, wherein R₂ is C₁₋₆ alkyl substituted with a moiety of formula (i).

10. The compound of claim 9, wherein R₁, R₃, R₄, and R₅ do not comprise a polyethylene glycol moiety.

11. The compound of claim 2, wherein R₃ is C₁₋₆ alkyl substituted with a moiety of formula (i).

12. The compound of claim 11, wherein R₁, R₂, R₄, and R₅ do not comprise a polyethylene glycol moiety.

13. The compound of claim 2, wherein R₄ is C₁₋₆ alkyl substituted with a moiety of formula (i).

14. The compound of claim 13, wherein R₁, R₂, R₃, and R₅ do not comprise a polyethylene glycol moiety.

15. The compound of claim 2, wherein R₅ is a moiety of formula (i).

16. The compound of claim 15, wherein R₁, R₂, R₃, and R₄ do not comprise a polyethylene glycol moiety.

17. The compound of claim 1 having the structure of formula (II): 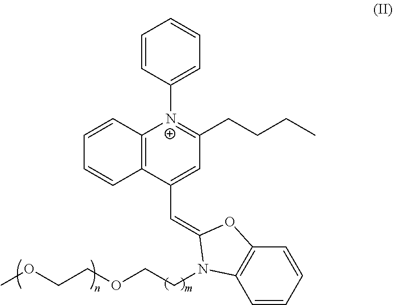
wherein n is 1-10, inclusive; and
m is 1-5, inclusive.

18. The compound of claim 1 having the structure of formula (III): 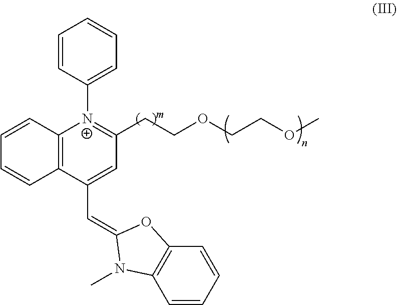
wherein n is 1-10, inclusive; and
m is 1-5, inclusive.

19. The compound of claim 1 having the structure of formula (IV): 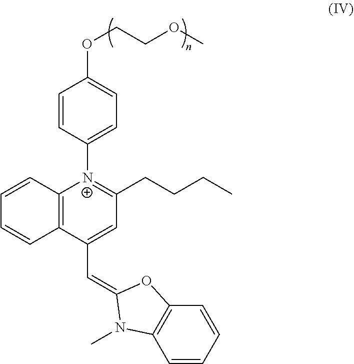
wherein n is 1-10, inclusive.

20. The compound of claim 1 having the structure of formula (V): 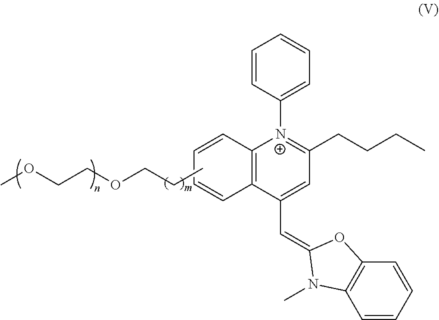
wherein n is 1-10, inclusive; and
m is 1-5, inclusive.

21. The compound of claim 1 having the structure of formula (VI): 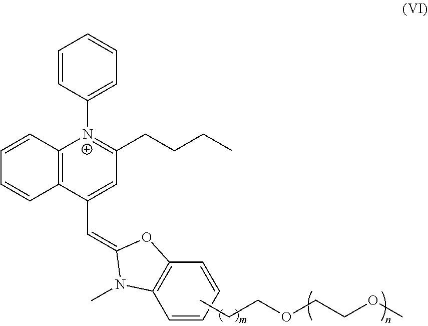
wherein n is 1-10, inclusive; and
m is 1-5, inclusive.

22. The compound of claim 1, selected from:

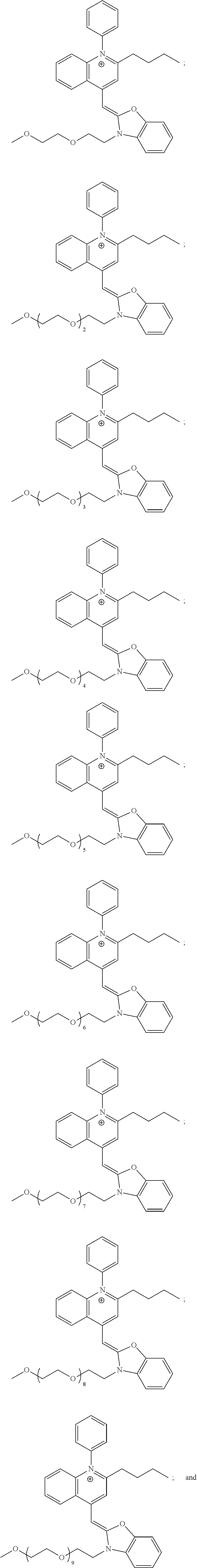

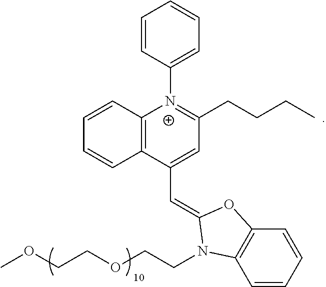

23. A method of detecting the presence or absence of an analyte in a test sample, comprising contacting the test sample with a compound of claim 1 in the presence of an energy (E₁) and detecting emission of an energy (E₂) from the sample.
